(19) United States
(12) Patent Application Publication
(10) Pub. No.: US 2021/0338892 A1
(43) Pub. Date: Nov. 4, 2021

(54) AUTOMATED METHOD FOR TREATING A BIOLOGICAL TISSUE IN ORDER TO PRODUCE A TISSUE MATRIX

(71) Applicant: Theracell Consulting Sprl, Lasne (BE)

(72) Inventor: Denis Dufrane, Lasne (BE)

(21) Appl. No.: 17/269,651

(22) PCT Filed: Aug. 23, 2018
(86) PCT No.: PCT/EP2018/072741

§ 371 Date: Feb. 19, 2021

(51) Int. Cl. A61L 27/36; C12N 5/071; B01L 3/00; B01L 9/06

(52) U.S. Cl. CPC A61L 27/3691; C12N 5/0697; B01L 2300/0832; B01L 9/06; B01L 2300/0609; B01L 3/5021

(57) ABSTRACT

The invention concerns an automated method for treating a biological tissue in order to produce a tissue matrix for allograft or xenograft, wherein the following sequence of steps: a) introducing said tissue into a treatment solution, b) treating the tissue in the solution, while stirring, c) removing the tissue from the treatment solution and d) emptying the reactor vessel, is repeated until the end of the treatment, and wherein all of the steps of all of the sequences are implemented automatically in a “classified” enclosure and inside a single reactor vessel, by changing the treatment solution in the reactor vessel between two sequences. The reactor vessel includes a container designed to contain a biological tissue treatment solution into which a removable biological tissue receiver and stirring means are introduced.

This application concerns the field of the production of human or animal tissues, with a view to use them for grafts (allografts or xenografts).

An allograft involves a human recipient and a donor. An autograft involves a single human-being.

A xenograft involves an animal donor and a human recipient.

A growing number of human musculoskeletal disorders require treatments by bone replacement or reconstruction, in the dental field (maxillo-facial reconstruction by dental implant), as well as in oncology (following the ablation of bone tumours) or in orthopaedic surgery (following a fracture or for the fusion of vertebrae). At present, bone reconstruction is done by autograft, usually by puncturing bone at the level of the iliac crest of the person who needs to be treated. However, not only is this puncture painful, but also irreversible. In fact, the hip bone does not regenerate, and the quantity of the available bone tissue is thus limited. The autograft also requires a double surgical intervention on the same individual, which also has a significant impact on post-operative recovery.

Another approach is the use of bone substitutes, which are widely accessible and inexpensive, e.g. ceramic, coral or glass prostheses. Unfortunately, due to the poor regulation in this market, many problems relating to reliability, material compatibility, and even toxicity cause many post-operative problems and the medical staff are increasingly losing trust in these substitutes.

Allografting is also practiced. In practice, bone tissue is removed from an individual, for example a femoral head removed during the fitting of a hip implant. This tissue contains the bone matrix as well as donor cells. In order to eliminate the risk of rejection, or inflammatory response, during transplantation, it is necessary to treat the tissue in order to clean it of all “genetic” traces of the donor, i.e. to eliminate the traces of cells, blood or fat from it. The method of cleaning or “decellularising” the bone tissue to provide a decellularised bone matrix is described in Dufrane et al., Biomaterials. 2002 July; 23(14):2979-88 and Dufrane et al. Eur Cell Mater. 2001 Jan. 10; 1:52-8; discussion 58.

The main steps involved in this cleaning method are:
Centrifugation of the tissue taken from the donor to remove blood and fat from it;
Cutting the tissue, usually into cubes of varying size;
Treatment in one or more solutions, in order to remove the traces of cells from it, and to inactivate viruses and/or bacteria.

After cleaning, the next step is freeze-drying to obtain the stable demineralised bone matrix followed by packing.

Although allografting is presently the most reliable bone reconstruction technique, patient access to this technique is highly restricted by the method and the cost of producing the decellularised bone matrix. The applicant has developed an automated method to carry out these steps in a classified sterile chamber. This method is described in WO2018122039. This process for obtaining a tissue matrix for allograft or xenograft, includes, after collecting a biological tissue, the steps according to which:
the tissue is classified,
the tissue is possibly cut,
the tissue is treated and
the obtained tissue matrix is packed,
   all these steps are implemented in an automated manner inside a single “classified” reaction vessel. The main advantages of this process are ensuring the traceability of the batches as regards the origin of the tissue, particularly by avoiding possible human errors, and considerably reducing the production cost of tissue matrices.

If the steps of classification, cutting and packing are rather short, i.e. lasting only a few minutes, the step of treating the tissues can last several hours and is therefore likely to limit the productivity of the automaton implementing the process.

Indeed, a tissue treatment involves stirring the tissue in several successive baths. Therefore, once the tissue has been cut into pieces, it is necessary to introduce the pieces into a bath, to stir it for a certain amount of time, to remove the tissue from the bath and introduce it into the next bath, to stir it again for a determined period of time, and so on until the treatment is complete. The number of successive baths can vary depending on the nature of the tissue. Moreover, each tissue must be treated individually in order to avoid cross-contamination.

To maintain a high rate of tissue treatment, it is then necessary to simultaneously process several (even a large number of) treatment lines each including a series of vessels containing the solutions in which the tissue pieces must be successively immersed. This involves having a large number of treatment vessels available. Particularly, if we consider that these vessels must be replaced between each tissue batch, it is necessary to introduce a large stock of these vessels inside the classified chamber. This way of working is neither ecological nor cheap.

These production problems are not limited to bone tissue, and are also encountered for other biological tissues used in allografts, like skin, tendons, bladder or adipose tissue. These same problems are also encountered for the production of cell therapies or during the treatment of cells derived from tissues. In order to improve the efficiency of automated tissue treatment, the applicant has developed a method and a support that solves the problems stated above.

### Solution of the Invention

This invention offers, for this purpose, an automated process for the treatment of a biological tissue to produce a tissue matrix for allografts or xenografts according to the following sequence of steps:
a) the tissue is introduced into a treatment solution,
b) the tissue is treated in the solution, with stirring,
c) the tissue is removed from the treatment solution,
d) the reaction vessel is emptied,
   repeated till the end of the treatment, characterised by the fact that all the steps of all the sequences are implemented in an automated manner in a “classified” chamber and inside a single reaction vessel, by changing the treatment solution in the reaction vessel between two sequences.

Biological tissue also refers to the pieces of biological tissues, i.e. a biological tissue that has already undergone a cutting step. The process of the invention, carried out in a “classified” chamber, is carried out under sterile conditions that satisfy very specific standards.

To implement the process of the invention, a reaction vessel is also proposed which includes:
a container arranged to contain a treatment solution for the biological tissue into which is introduced
a receptor for the biological tissue and
means of stirring,
   characterised by the fact that the receptor is detachable.

Advantageously, the means of stirring are associated to the receptor, which enables manufacturing only one element having the two functions of stirring and supporting biological tissue.

Thus, the biological tissue on its receptor can be introduced into the reaction vessel containing a treatment solution to carry out step a) of the process of the invention; the means of stirring allow the implementation of step b) of treatment with stirring; as the receptor is detachable, it makes it possible to extract the biological tissue from the solution according to step c); the vessel can be emptied of its solution according to step d) and the sequence of steps can be repeated. A single reaction vessel enables carrying out all the steps of the treatment. It thus makes it possible to reduce the bulk in the treatment chamber. A large number of batches can thus be treated at the same time in the classified chamber, thus reducing the congestion resulting from the relatively long duration of the treatment compared to the other steps of introduction, cutting, packing, etc.

The detachable receptor is arranged so as to be at least partially permeable to liquids, i.e. perforated so as to act as a support to the biological tissue and to allow it to be immersed in the treatment solution in step a) and to drain it when it is extracted from the container in step c).

As each batch of tissue is kept in the same vessel throughout the treatment, its traceability is ensured, along with the absence of cross-contamination.

Advantageously, the treatment process of the invention can be implemented more specifically in the process for obtaining a tissue matrix for allografts or xenografts, in an automated manner inside a single “classified” reaction vessel, according to which, after collecting a biological tissue:
the tissue is classified,
the tissue is cut,
the tissue is treated and
the obtained tissue matrix is packed,
   in which to treat the tissue, the sequence of the following steps are repeated till the end of the treatment:
a) the tissue is introduced into a treatment solution,
b) the tissue is treated in the solution, with stirring,
c) the tissue is removed from the treatment solution,
d) the reaction vessel is emptied,
   with all the steps of all the sequences of the treatment being implemented inside a single reaction vessel by changing the treatment solution in the reaction vessel between two sequences.

For the implementation of this process, the reaction vessel of the invention is advantageously included in a kit containing a plate supporting:
the reaction vessel of the invention, and
a receptacle for the tissue to be treated.

The kit can also include
fingers for gripping the biological tissue, arranged to be mounted on a tissue cutting automaton and/or
fingers for gripping the biological tissue, arranged to be mounted on a tissue moving automaton.

Automaton refers to an automated means, such as a robot or a robotic arm.

The receptacle for the tissue to be treated advantageously includes the means for positioning the tissue. This enables, right from the introduction of the tissue into the classified reaction vessel, it to be positioned so as to be optimally supported by the automatons carrying out the different steps of the tissue matrix obtaining process.

The fingers for gripping the biological tissue, arranged to be mounted on the tissue cutting automaton must be capable of holding the tissue in place during the cutting step that may involve a high-pressure water jet.

Thus, a kit corresponds to a batch of tissue to be treated. It accompanies this tissue right from its introduction into the chamber till the final packing, with the elements of the kit being used and then possibly thrown after use. This kit is thus preferably disposable.

The kit may also include the final container(s) in which the tissue matrix, possibly in pieces, will be packed.

The tissue treatment solution can be a chemical treatment or a biological treatment solution. The type of solution to be used depending on the tissue to be treated is known to the person skilled in the art.

A treatment can be a chemical treatment including exposing the biological tissue to chemical agents or reagents, e.g. stirring the tissue in a solution containing an antibacterial agent, an organic solvent or a solution whose pH is adjusted to be acidic or basic, by adding ionic compounds.

A biological treatment refers to the application of biological agents to the tissue concerned, like, for example, proteins or growth factors inducing a cell differentiation.

The biological tissue can undergo several consecutive treatments that have different purposes, like, for example, the degreasing or inactivation of bacteria or viruses or prion.

It is emphasised here that the automated process of treating a biological tissue, the process of obtaining a tissue matrix, the reaction vessel and the kit are linked by the same inventive concept, which is the reduction of the volume of equipment inside the chamber and the reduction of the space-time required for the treatment to allow the tissue matrix production line to operate continuously, despite the steps of very different durations.

The invention will be better understood with the help of the following description of several implementations of the invention, with reference to the appended drawing, in which:

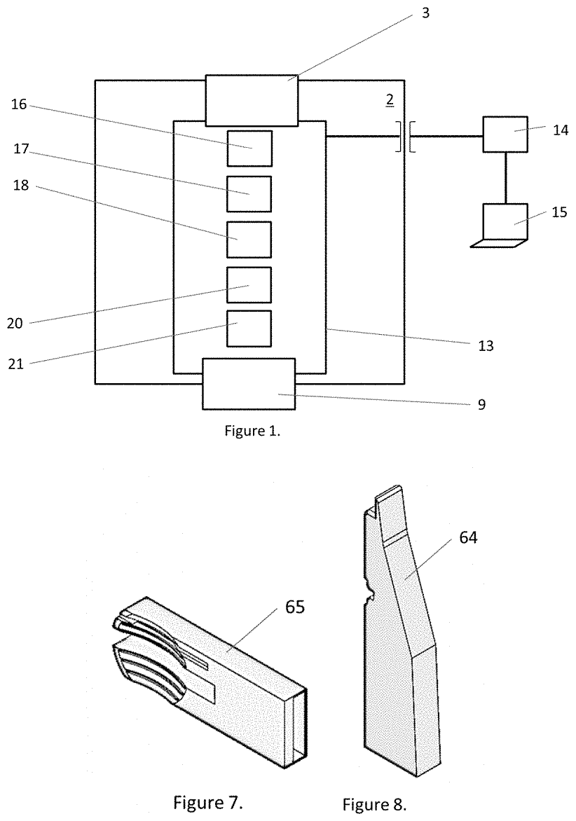

FIG. 1 is a schematic illustration of a sterile chamber for the implementation of the invention,

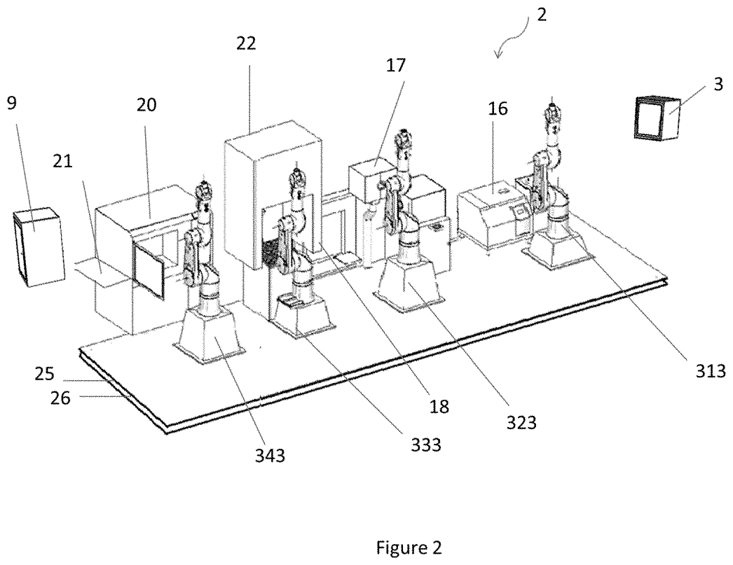

FIG. 2 represents, in perspective, the inside of a sterile chamber for the implementation of the process of the invention,

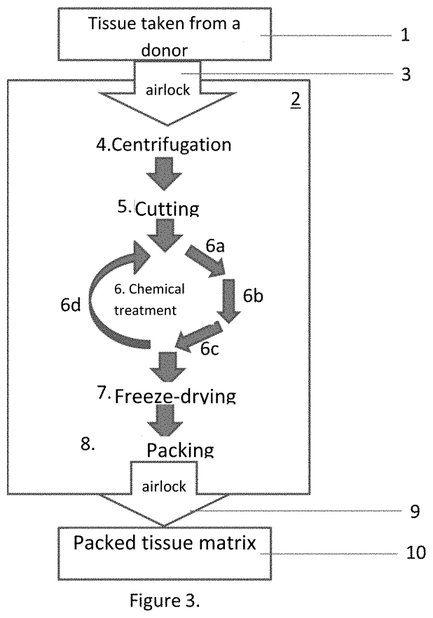

FIG. 3 is a schematic illustration of the process of the invention,

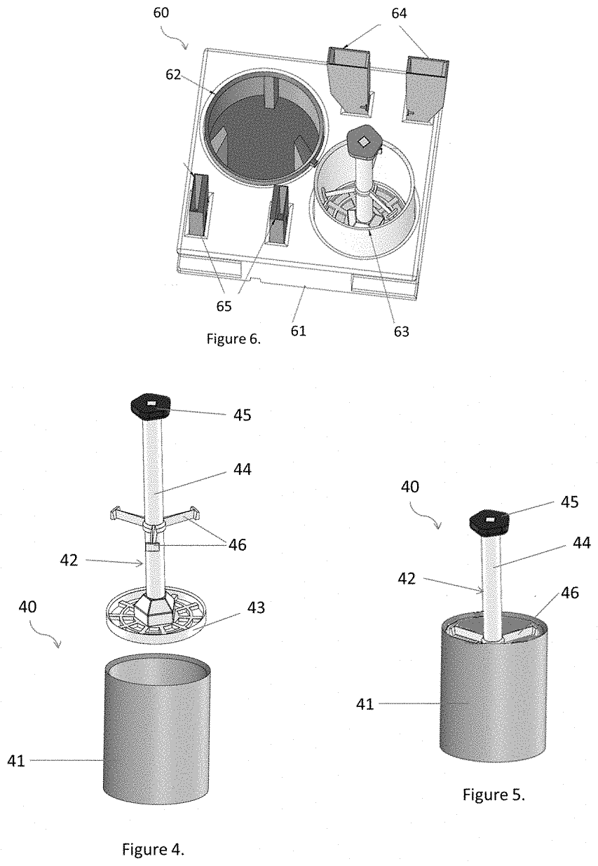

FIGS. 4 and 5 are illustrations in perspective of a reaction vessel according to the invention, in two different positions,

FIG. 6 is an illustration, in perspective, of a kit according to the invention,

FIG. 7 is an illustration, in perspective, of a finger for gripping of the kit of FIG. 6 and

FIG. 8 is an illustration, in perspective, of another finger for gripping of the kit of FIG. 7.

With reference to FIGS. 1 and 2 illustrating the process and the reaction vessel described in WO2018122039, inside reaction vessel 2, a series 13 of automatons 313, 323, 333 and 343, cooperating with each other, is arranged to enable the implementation of the steps of the process. This series of automatons 13 is advantageously controlled by a computer system 14 to which instructions may have been given by an operator, by means of a control station 15, here a computer.

Reaction vessel 2 includes an airlock 3 for the introduction and classification of biological tissues, taken from a donor, and an airlock 9 for the outlet of packed tissue matrix, and each airlock here is placed at one end of the reaction vessel. Here, there are five pieces of equipment, placed side by side, between the two airlocks: a centrifuge 16, a cutting unit 17, a chemical treatment station 18, placed under a hood 22, a freeze-dryer 20 and a packing station 21, in front of which are respectively positioned the automatons, which here are the four articulated robotic arms 313, 323, 333 and 343. All the elements described above are placed on a floor 25 under which a space 26 is provided.

The elements of reaction vessel 2 having been described, the articulation of the elements between them for the implementation of the process of the invention will now be explained in detail for a tissue that may, for example, be a femoral head, taken from a donor individual, during the fitting of a hip implant. In order for this raw femoral head to be used for the production of bone matrix with a view to its use for a bone reconstruction in another patient, it must undergo a certain number of transformations, in a controlled sterile environment.

After opening the external door of airlock 3, an operator puts a biological tissue to be treated, in an appropriate receptacle, in this airlock. The airflow inside the airlock is such that no external contamination can enter the airlock until the external door is closed.

The automated process for obtaining cell matrix starts with the opening of the internal door (to reaction vessel 2) of airlock 3.

The robotic arm 313 grips the receptacle containing the biological tissue in airlock 3 and deposits it in a cavity of the centrifuge 16.

The centrifugation separates the bone matrix from unwanted residues, such as marrow, blood or fatty residues. The femoral head, which is thus cleaned, is then picked up by the cutting automaton 323 which moves it to the cutting unit 17, configured to produce pieces of bone of a predefined size. Here, cutting by a non-abrasive high-pressure water jet is carried out in order to, on the one hand, ensure very precise and clean cutting, without an increase in temperature that could damage the tissues, and, on the other hand, not contaminate the batches amongst themselves.

In practice, the arm 323 holding the biological tissue to be cut in place is mobile along several axes simultaneously and positions and moves the tissue to be cut under the jet, according to any predefined direction. As the articulated arm is mobile along several axes, it is thus possible to obtain any cutting geometry, which makes it possible to optimise the cutting in order to limit waste and obtain a higher yield of tissue matrix at the end of the process.

These pieces are then collected by the arm 333 and introduced into a treatment area 18, where they will undergo several successive sequences of steps of immersion and stirring in treatment solutions, possibly supplemented by one or more rinsing steps. This treatment step will be described in more detail below.

The robotic arm 333 collects the pieces of biological tissue treated one-by-one to place each of them in a packing container, like, for example, a pre-sealed tube, i.e. a tube whose caps are already placed without however being sealed, and then introduces each tube in the freeze-dryer 20. At the end of the freeze-drying, before reopening the freeze-dryer, the tubes are sealed, for example, using a plate that presses on the caps in order to seal the tubes. Each tube thus contains a piece of tissue matrix, which is ready to be used for a graft.

The arm 343 will then complete the packing of the tubes, for example by placing a label on them that states all the information necessary for the traceability of the batch, and then place the packed tissue matrix tubes in the outlet airlock 9 for them to be collected by an operator.

The actions performed here by the robotic arms 333 and 343 could be performed by a single arm, or other automated arms could be added. It is important to note here that the precise articulation of the different tasks carried out by the robotic arms is very flexible and any other relevant arrangement, with more or less arms, can be considered.

The freeze-drying enables drying the tissue matrix until it contains, for example, less than 5% moisture, and can be vacuum-packed or packed in a protective atmosphere in a packing unit 21. This provides optimal stability to the bone matrix allowing it to be stored at ambient temperature for several months. It is possible to include a step of labelling the packing in order to ensure the traceability of the batches produced. The packed tissue matrix can then be extracted from reaction vessel 2 through outlet airlock 9, similar to inlet airlock 3. It is even possible to configure the reaction vessel 2 so that there is only one inlet and outlet airlock.

The command and control station 15 comprises the computer system 14 in which the manufacturing process of the packed tissue is entered. The computer system 14 controls the series 13 of automatons in reaction vessel 2. Some parameters may have to be entered by the operator, like, for example, the nature of the tissue, whether the automaton can manage the production of several types of tissue, the durations of the chemical treatments or the percentage of moisture desired in the packed tissue matrix.

The manufacturing process described above thus enables introducing a “raw” biological tissue into a reaction vessel and collecting, at the outlet, a biological tissue that has been decontaminated and is ready to be used for a graft. There is only one classification step in this process, when tissue 1 enters reaction vessel 2. The continuity of the classification is ensured by the restricted and controlled space of the reaction vessel from which the tissue does not come out until the end of production. As no operator has to intervene while the process is being carried out, errors during handling are eliminated, traceability is ensured, and the requirements in terms of space and handling are reduced to the bare minimum.

Nevertheless, the treatment step is long as compared to the other steps that the biological tissue must undergo. This step must thus be optimised and integrated into the overall tissue matrix production process so as to not limit its effectiveness and to optimise the operation time of each element/device contained in the chamber.

With reference to FIG. 3, the steps of the process for obtaining tissue matrix 10 are centrifugation 4 of tissue 1, cutting 5 of this tissue, treatment 6, freeze-drying 7 and packing, all operations carried out in a sterile chamber 2 provided with an airlock 3 to introduce tissue and an airlock 9 to take out the tissue matrix. Step 6 of the treatment includes several sub-steps. In step 6a, the tissue cut in pieces is introduced into a treatment solution inside a reaction vessel, and then in step 6b, the tissue pieces are treated in the solution, i.e. the tissue is stirred for a pre-determined duration in the treatment solution. Then, in step 6c, the tissue is removed from the treatment solution, and in step 6d, the reaction vessel is emptied of the solution that it contains. The sequence of the steps 6a to d is repeated until the end of the treatment, i.e. that a new treatment solution, of the same composition or a different composition, is introduced into the same reaction vessel as was used in the first sequence, the pieces of tissue are introduced into it, stirred for the required time and then taken out from it.

When all the sequences necessary for the treatment have been completed, the pieces of tissue can then be freeze-dried and packed.

It is thus understood that a batch of biological tissue is treated in a vessel called a “reaction vessel”. This reaction vessel is manufactured so as to be able to successively contain a variety of treatment solutions, which are generally chemical treatment solutions. There are no multiple vessels each containing a type of solution in which the tissue would be successively immersed. This saves a considerable amount of space in the sterile chamber. This also enables simultaneously initiating the treatment of several batches of tissue, one after the other, by limiting the number of vessels to be stored in the treatment space in the sterile chamber. For example, as soon as the cutting of the first batch is completed, the pieces are introduced into a first reaction vessel and several successive sequences of treatment steps 6a to d in several treatment solutions, successively introduced into the first reaction vessel, will start. During this time, a second batch of tissue can be cut into pieces, which are then introduced into a second reaction vessel and the successive sequences of the treatment steps 6a to d are also initiated in the second reaction vessel. Thus, the treatments of multiple batches can be initiated successively and simultaneously, i.e. in the same space, but staggered in time. As soon as the treatment of the first batch is complete, the pieces of tissue of the first batch are sent to the freeze-drying step, thereby freeing up space to start treating a new batch, in its reaction vessel. Ideally, the number of batches that can be treated in the space dedicated for the treatment of the sterile chamber is adjusted according to the time required for the treatment compared to the time required for the other steps, like, for example, cutting or freeze-drying. The equipment installed in the sterile chamber can thus ideally operate continuously.

In order to carry out such a treatment, the reaction vessel must have certain essential features. It must mainly enable a treatment solution to be introduced into it, for the pieces of tissue to be immersed in it, for them to be stirred in it, and then for them to be removed from it and for the solution to be emptied from the vessel. Referring to FIGS. 4 and 5, such a reaction vessel 40 comprises a container 41, which is cylindrical here, and a receptor 42 of biological tissue. Here, receptor 42 has a perforated circular base 43, the diameter of which is slightly less than the diameter of container 41. The base 43 is placed at the end of a rod 44 and the other end of this rod has a nut head 45. At the middle of the rod, there are three radially arranged blades 46, forming an angle of 130° between them. The receptor 42 is separate from the container 41 but is shaped to be inserted in it. When the receptor 42 is inserted in container 41, only a part of the rod 44 and the coupling nut 45 come out from the container 41.

In practice, for the treatment in question here and with reference to the chamber of FIG. 2, after the arm 333 has collected the pieces of cut tissue, it deposits them on the base 43 of the reaction vessel 40, i.e. when the receptor is inserted in the container, either when it is taken out of it, or partially taken out. At this stage, the reaction vessel may already be present in the treatment area 18 or else be introduced in it only when the pieces of tissue have already been deposited on the receptor 42.

The first treatment solution is introduced into the container 41, either when the receptor 42 supporting the tissue is already introduced in it, or, and preferably, before its introduction. In the latter case, the receptor 42 supporting the tissue is then immersed in the treatment solution contained in the container 41, enabling the tissue to be immersed in it. The receptor 42 is then rotated, using the coupling nut 45. The rod 42, the base 43 and the blades 46 are then driven in a rotary movement, with the blades 46 being provided with blades at their ends, they enable the homogenisation of the treatment solution. The coupling nut 45 enables the robotic means of the treatment area 18 to grip the receptor 42 to give it the rotational movement required for the stirring. It can also allow the same, or other, robotic means to give the receptor 42 a vertical shifting movement to introduce the receptor 42 into the container 41 or to come out of it. When the tissue has been sufficiently stirred, the robotic means lift the receptor 42 out of the container 41. The perforated structure of the base 43 allows drying the pieces of tissue. The container 41 can then be emptied of the treatment solution it contains. The solution can, for example, be poured out by tilting or turning the container 41, or it can be sucked out by automated means provided in the treatment area, or even a valve outlet can be provided at the bottom of the container which would be activated by automated means provided in the treatment area.

If another sequence of treatment steps is provided for, the container 41 can be rinsed, if need be, before introducing another treatment solution, in order to avoid adverse reactions.

Alternatively, it is possible that a sequence of treatment steps be a rinse sequence, in which case a rinsing solution is introduced into the container 41, and the receptor 42 supporting the tissue pieces is reintroduced into the container 41 and the stirring is started for a determined duration before again removing the receptor 42 from the container 41 and emptying the rinsing solution from it. A new treatment sequence can then begin in another treatment solution.

If the treatment is finished, the drained tissue pieces are then picked up by the robotic arm 333 for freeze-drying. The reaction vessel 40 can then be discarded, or set aside to be recycled. Thus, a reaction vessel is used only once, for a single batch of tissue. This is to avoid mixing tissues from different batches and/or transmitting contaminations from one batch to another.

One may consider organising the treatment area 18 as a carousel where the reaction vessels of the arriving batches move gradually along a “chain” where the various steps of the successive sequences can be carried out, with the tissue batch in its reaction vessel thus going through a sort of treatment loop, where new reaction vessels containing new tissue batches to be treated can be continuously deposited in this chain/loop.

Handily, the reaction vessel 40 can be part of a kit including single-use material components, intended to accompany the biological tissue during the steps that it needs to undergo before obtaining a tissue matrix. The kit mainly enables facilitating the introduction of the biological tissue through the airlock 3, improving its traceability and avoiding cross-contamination between batches of tissue.

Referring to FIG. 6, a kit 60 includes a plate 61 supporting a tissue receptacle 62, a reaction vessel 63, two pairs 64 and 65 of fingers for gripping the biological tissue. Here, the reaction vessel 63 corresponds to the reaction vessel illustrated in FIGS. 4 and 5.

Here, the receptacle 62 is arranged with three studs 66 intended to position a femoral head in a particular manner here, for its introduction into the sterile chamber. Thus, the femoral head is optimally positioned for the first robotic arm to pick it up. Depending on the nature of the tissue from which the tissue matrix is to be obtained, the receptacle 62 can be arranged differently and in any suitable manner.

The receptacle 62 can be arranged to be able to be introduced directly into the centrifuge. For example, a robotic arm collects the plate 61 of kit 60, takes the receptacle 62 containing the biological tissue there, introduces the receptacle into the centrifuge, comes out of it at the end of the centrifugation and puts the receptacle back on the plate 61 before taking kit 60 to the next step. For example, the kit is placed on an intermediate rack while waiting for it to be picked up by the next robotic arm, or for the robotic cutting arm to just take the tissue from it.

Advantageously, the receptacle is arranged to separate the tissue from the fluids expelled during a centrifugation. It can, for example, include a screen supporting the tissue and separating it from the bottom of the receptacle so that the fluids expelled from the tissue during the centrifugation are collected at the bottom, but are not in contact with the tissue.

The cutting arm, before grasping the biological tissue, which here is the femoral head will have gripping fingers, which here is the pair 65, specifically adapted to be able to subject the biological tissue to a high-pressure water jet, without these fingers releasing the biological tissue under pressure. The structure or material used for these gripping fingers will obviously differ depending on whether the tissue is skin or bone, soft tissue or hard tissue. They can, for example, include pins enabling to hold the tissue during the cutting, or grooves, as illustrated in FIG. 7. These fingers thus include the means to grip a biological tissue.

Depending on the robotic arms used, there may be more than two fingers.

At the end of the cutting, the fingers are removed from the robotic arm to be discarded, or put back on the plate 61 of the kit 60, or even set aside to be recycled. The gripping fingers arranged to be mounted on the arm/cutting automaton are thus disposable, like gloves. This prevents the contamination between the different tissue batches. This feature is particularly interesting in combination with cutting using a high-pressure water jet, which also avoids the possibilities of cross-contamination that are inevitable when one uses blade or strip cutting systems, of which the cutting components cannot be replaced between two batches.

The treatment step is then carried out using the reaction vessel 63, as explained in detailed above.

The other pair 64 of gripping fingers is intended for the robotic arm(s)/automaton(s) that pick up the pieces of tissue, mainly to move them, for example, just after cutting it or after the treatment. The gripping fingers for movement are of a design that is different from the pair for the cutting step, and can, for example, look like the finger shown in FIG. 8. The same gripping fingers can be used by one or more arms to pick up the same batch of biological tissue. Like for cutting, this avoids contamination between batches.

The gripping fingers are described here as a part of the kit. Depending on the configuration of the chamber, the fingers can be stored inside the chamber, regardless of the kit, and taken from this stock as may be needed. In other configurations, there can be a provision for the fingers to be cleaned and/or disinfected between each batch inside the chamber.

If required, there can be a provision for the tissues of different types to be treated successively in the sterile chamber. In this case, each tissue is introduced into a kit including the elements (receptacle, fingers, reaction vessel) suitable to its specific treatment. As the kit follows the tissue throughout the steps of obtaining the tissue matrix, and providing the elements that are specific to it, the internal equipment of the sterile chamber would thus not need to be changed to successively treat tissues of a different type. Thus, all that remains is entering the computer parameters managing the steps carried out in the chamber.

The elements of the kit are advantageously all marked with a tracking id of the tissue batch to be treated, like, for example, a number, barcode or a QR code. The kit could also contain other items such as the packing of the final tissue matrix pieces.

The plate 61 of the kit is advantageously shaped to be supported by the equipment inside the sterile chamber, and/or to be temporarily stored there on a rack or shelf.

The kit is advantageously a disposable kit that is discarded and/or decontaminated after use to be recycled.

Although the invention has been described here with the example of a bone matrix obtained from a femoral head, it can be applied to other biological tissues, like, for example, those from other bone sources or from soft tissues like the fascia lata.

1. An automated process for the treatment of a biological tissue to produce a tissue matrix for allografts or xenografts, wherein the sequence of steps is as follows:
a) the tissue is introduced into a treatment solution,
b) the tissue is treated in the solution, with stirring,
c) the tissue is removed from the treatment solution,
d) the reaction vessel is emptied,
wherein the sequence of steps is repeated till the end of the treatment, characterised in that all the steps of all the sequences are implemented in an automated manner in a “classified” chamber and inside a single reaction vessel, by changing the treatment solution in the reaction vessel between two sequences.

2. A process for obtaining a tissue matrix for allografts or xenografts, in an automated manner inside a single “classified” reaction vessel, according to which, after collecting a biological tissue:
the tissue is classified,
the tissue is cut,
the tissue is treated and
the obtained tissue matrix is packed,
wherein the tissue is treated by the sequence of the following steps, which are repeated till the end of the treatment:
a) the tissue is introduced into a treatment solution,
b) the tissue is treated in the solution, with stirring,
c) the tissue is removed from the treatment solution,
d) the reaction vessel is emptied,
with all the steps of all the sequences of the treatment being implemented inside a single reaction vessel by changing the treatment solution in the reaction vessel between two sequences.

3. The process according to claim 2, further comprising the steps of:
centrifuging the biological tissue, and/or
freeze-frying the tissue matrix before packing.

4. A reaction vessel comprising:
a container arranged to contain a treatment solution for the biological tissue into which is introduced
a receptor for the biological tissue and
means of stirring,
characterised in that the receptor is detachable.

5. The reaction vessel according to claim 4, in which the detachable receptor comprising a perforated support base for the biological tissue.

6. The reaction vessel according to claim 4, in which the means of stirring are connected to the receptor.

7. The reaction vessel according to claim 5, in which the means of stirring comprises at least one blade that is radially arranged along a rod, one end of which is the perforated support base for the biological tissue.

8. The reaction vessel according to claim 7, of which the container is arranged to contain at least the part of the rod on which the blade and the base are arranged.

9. A kit comprising a plate supporting:
the reaction vessel according to claim 4 and
a receptacle for the tissue to be treated.

10. The kit according to claim 9, also comprising fingers for gripping the biological tissue, arranged to be mounted on a tissue cutting automaton and/or fingers for gripping the biological tissue, arranged to be mounted on a tissue moving automaton.

11. The kit according to claim 9, in which the receptacle for the tissue to be treated includes the means for positioning the tissue.

12. The kit according to claim 9, in which the receptacle is arranged to separate the tissue from the fluids expelled during a centrifugation.

13. The kit according to claim 9, in which the fingers for gripping the biological tissue, arranged to be mounted on a tissue cutting automaton, include the means to grip a biological tissue.
